(19) United States
(12) Patent Application Publication
(10) Pub. No.: US 2026/0306793 A1
(43) Pub. Date: Oct. 8, 2026

(54) CATHETER WITH INFLATABLE EXPANSION TUBE STRUCTURE

(71) Applicant: Charles Mansour, Fairhope, AL (US)

(72) Inventor: Charles Mansour, Fairhope, AL (US)

(21) Appl. No.: 19/569,414

(22) Filed: Mar. 17, 2026

Related U.S. Application Data
(60) Provisional application No. 63/785,160, filed on Apr. 8, 2025.

(51) Int. Cl. A61M 25/00; A61F 2/00; A61M 39/00; A61M 39/24

(52) U.S. Cl. CPC A61M 25/0045; A61F 2/0027; A61M 25/0023; A61M 39/24; A61F 2230/0006; A61F 2230/0069; A61F 2230/0091; A61F 2250/0003; A61F 2250/001; A61M 2025/0024; A61M 2025/0025; A61M 2039/0009; A61M 2039/244; A61M 2205/0216; A61M 2205/3331; A61M 2210/1085; A61M 2210/1096

(57) ABSTRACT

A catheter for conveying fluids within the body and a method of using the catheter are provided. The catheter has an inflatable and deflatable expansion tube structure that allows the catheter to transition between an inflated configuration and a deflated configuration. When the catheter is in the inflated configuration, the lumen of the catheter is expanded to a maximum diameter to allow the flow of fluid or the insertion of instrumentation. When the catheter is in the deflated configuration, the lumen is substantially occluded to restrict the flow of fluids. The expansion tube structure may have a helical tube structure or a series of individual tubular rings each fluidly connected to an adjacent tubular ring by an inflation tube. The expansion tube structure may include a series of one-way valves that open and close depending on whether the catheter is in the inflated configuration or deflated configuration.

### FIELD OF THE INVENTION

The present disclosure relates generally to a catheter for conveying fluids within the body. The catheter has an inflatable expansion tube structure for occlusion and dilation of the lumen of the catheter.

### BACKGROUND

Catheters are widely used medical devices for various diagnostic and therapeutic procedures. These flexible tubes may be inserted into body cavities, vessels, or ducts to allow for the introduction or withdrawal of fluids, medications, or medical instruments. Catheters play a crucial role in numerous medical applications, including urinary drainage, intravenous therapy, cardiovascular interventions, and minimally invasive surgeries. Traditional catheters typically have a fixed diameter, which can limit their versatility in certain medical scenarios. In some situations, medical professionals may need to adjust the catheter's size or shape to accommodate different anatomical structures or to perform specific procedures. This limitation has led to the development of various specialized catheters for different applications, resulting in increased inventory and costs for healthcare providers.

Additionally, the insertion and removal of catheters can sometimes cause discomfort or trauma to patients, particularly when navigating through narrow or sensitive anatomical passages. There is an ongoing effort in the medical field to develop catheters that can be more easily inserted and removed while minimizing patient discomfort and potential tissue damage. Furthermore, in certain medical procedures, it may be beneficial to have a catheter that can selectively allow or restrict fluid flow or instrument passage. This capability can enhance the catheter's functionality in applications such as targeted drug delivery, temporary vessel occlusion, or controlled drainage.

As medical procedures continue to advance and become more complex, there is a growing demand for innovative catheter designs that can adapt to various physiological conditions and procedural requirements. Improved catheter technologies have the potential to enhance patient outcomes, reduce procedural complications, and increase the efficiency of medical interventions.

### SUMMARY

In one aspect, a catheter having an inflatable expansion tube structure disposed within the catheter is provided. The inflatable tube structure allows the catheter to transition between an inflated configuration and a deflated configuration. When the catheter is in the inflated configuration, the lumen of the catheter is expanded to its maximum diameter to allow the maximum flow of fluid through the lumen or to allow intraluminal passage of instrumentation into an organ system that dilates and constricts either pathologically or physiologically. When the catheter is in the deflated configuration, the lumen may be occluded to restrict the flow of fluids through the lumen. In some embodiments, the lumen may be substantially or entirely closed so that no fluid can flow through the lumen. In other embodiments, the lumen may be partially closed to restrict flow, and the catheter may be partially inflated to control the size of the lumen and thus control the flow of fluid through the lumen.

The catheter comprises a flexible outer tube and an expansion tube structure attached to or embedded within an interior wall of the outer tube. In some embodiments, the expansion tube structure may comprise a series of individual tubular rings or a helical tube structure. In a preferred embodiment, the expansion tube structure comprises a plurality of tubular rings disposed in spaced relation along a length of the outer tube. Each of the individual tubular rings defines an annular fluid chamber, and each tubular ring is attached to or embedded within the interior wall. The expansion tube structure further comprises an inflation tube fluidly connecting each tubular ring to an adjacent tubular ring such that the annular fluid chambers of all of the tubular rings are fluidly connected to each other. An injection port is connected to one of the tubular rings that is disposed at an end of the outer tube, and the injection port is configured to allow injection of a fluid, such as a sterile saline solution, into the annular fluid chambers of all of the tubular rings and connecting inflation tubes through the injection port to transition the catheter into the inflated configuration. The injection port is further configured to allow withdrawal of the fluid from the annular fluid chambers of all of the tubular rings through the injection port to transition the catheter into the deflated configuration. The lumen of the catheter may be defined by the outer tube and each of the plurality of tubular rings, each of which has a central opening that may in part define the lumen.

In a preferred embodiment, the catheter further comprises a flexible inner tube that is attached to each of the plurality of tubular rings. The inner tube extends through the central opening of each of the plurality of tubular rings. In this embodiment, the lumen may be defined by the inner tube, which may allow the partially or fully occluded lumen to more effectively control or restrict the flow of fluids through the lumen when the catheter is in the deflated configuration or a partially inflated configuration. The inner tube is preferably attached to each of the plurality of tubular rings by a plurality of short filaments or tethers. In this embodiment, the outer tube and the inner tube define an annular space disposed between the outer tube and the inner tube, and the attachment of the inner tube to the tubular rings by filaments allows any fluid that may be contained within the annular space to flow along the entire length of the catheter without becoming trapped in the annular space between adjacent tubular rings. In one alternative embodiment, the inner tube may be adhered directly to each of the plurality of tubular rings. In another alternative embodiment, an expandable rubber material may be disposed within the annular space such that the expandable rubber material substantially fills the annular space. In this alternative embodiment, the rubber material may contract when the catheter is in the deflated configuration, and the process of injecting fluid into the annular fluid chambers to transition the catheter into the inflated configuration may cause the rubber material to expand, thereby causing the lumen to dilate.

In another embodiment, the expansion tube structure comprises a helical inflation tube attached to or embedded within the interior wall of the outer tube in a helical configuration to define a helical fluid chamber. The injection port is connected to the helical inflation tube at an end of the catheter to facilitate transitioning the catheter between the inflated and deflated configurations. When fluid is injected into the helical fluid chamber to transition the catheter into the inflated configuration, the helical inflation tube expands outwardly to expand the diameter of the lumen, which may be defined by the outer tube and the helical inflation tube. When fluid is withdrawn from the helical fluid chamber to transition the catheter into the deflated configuration, the helical inflation tube contracts inwardly to constrict the diameter of the lumen to restrict the flow of fluid through the lumen. In this embodiment, the catheter may optionally include a flexible inner tube, which is preferably attached to the helical inflation tube by a plurality of short filaments or tethers disposed along the length of the inner tube. Alternatively, the catheter may comprise a pair of helical inflation tubes that form a double helix. In this case, the catheter may have a single injection port configured for injecting fluid into both helical inflation tubes simultaneously, or the catheter may have two injection ports configured for injecting fluid into each helical inflation tube separately.

In another embodiment, the catheter further comprises a plurality of valves each attached to an interior wall of the inner tube. Each of the plurality of valves is disposed in spaced relation along a length of the inner tube, and each valve comprises a pair of opposing flexible flaps configured to fit together to close the valve and to separate from each other to open the valve. When the catheter is in the inflated configuration, the inner tube may define the lumen, and the two opposing flaps of each valve may be separated to open the lumen to allow fluid flow through the lumen. When the catheter is in the deflated configuration, the two opposing flaps of each valve may contact each other to substantially close the lumen to prevent fluid flow through the lumen.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### DESCRIPTION OF THE DRAWINGS

These and other features, aspects, and advantages of the present invention will become better understood with regard to the following description, appended claims, and accompanying drawings where:

FIG. 1 shows a partial perspective view of a catheter with a series of inflatable rings and an inner tube in accordance with the present disclosure.

FIG. 2 shows a partial side view of a catheter with a series of inflatable rings and an inner tube in accordance with the present disclosure.

FIG. 3 shows a cross-sectional view of a catheter having a series of inflatable rings and an inner tube in an inflated configuration in accordance with the present disclosure.

FIG. 3A shows a cross-sectional view of a catheter having a series of inflatable rings and an inner tube in a deflated configuration in accordance with the present disclosure.

FIG. 4 shows a partial perspective view of an alternative embodiment of a catheter having a series of inflatable rings and an inner tube in accordance with the present disclosure.

FIG. 5 shows a partial side view of an alternative embodiment of a catheter with a series of inflatable rings and an inner tube with an expandable rubber material disposed outside of the inner tube in accordance with the present disclosure.

FIG. 6 shows a cross-sectional view of an alternative embodiment of a catheter with a series of inflatable rings and an inner tube in an inflated configuration with an expandable rubber material disposed outside of the inner tube in accordance with the present disclosure.

FIG. 6A shows a cross-sectional view of an alternative embodiment of a catheter with a series of inflatable rings and an inner tube in a deflated configuration with an expandable rubber material disposed outside of the inner tube in accordance with the present disclosure.

FIG. 7 shows a partial perspective view of a catheter with a series of inflatable rings and an inner tube adhered to the inflatable rings in accordance with the present disclosure.

FIG. 8 shows a partial side view of a catheter with a series of inflatable rings and an inner tube adhered to the inflatable rings in accordance with the present disclosure.

FIG. 9 shows a partial perspective view of a catheter with a series of inflatable rings in accordance with the present disclosure.

FIG. 10 shows a cross-sectional view of a catheter having a series of inflatable rings in accordance with the present disclosure.

FIG. 11 shows a partial perspective view of a catheter with a helical inflatable tube in accordance with the present disclosure.

FIG. 12 shows a partial side view of a catheter with a helical inflatable tube in accordance with the present disclosure.

FIG. 13 shows a partial perspective view of a catheter with two helical inflatable tubes forming a double helix in accordance with the present disclosure.

FIG. 14 shows a partial side view of a catheter with two helical inflatable tubes forming a double helix and having a single injection port in accordance with the present disclosure.

FIG. 15 shows a partial side view of a catheter with two helical inflatable tubes forming a double helix and having two injection ports in accordance with the present disclosure.

FIG. 16 shows a partial side view of a catheter with a series of inflatable rings and an inner tube with a series of valve flaps in an open position and attached to the inner tube in accordance with the present disclosure.

FIG. 17 shows a cross-sectional view of a catheter with a series of inflatable rings and an inner tube with a series of valve flaps in an open position and attached to the inner tube in accordance with the present disclosure.

FIG. 18 shows a partial side view of a catheter with a series of inflatable rings and an inner tube with a series of valve flaps in a closed position and attached to the inner tube in accordance with the present disclosure.

FIG. 19 shows a cross-sectional view of a catheter with a series of inflatable rings and an inner tube with a series of valve flaps in a closed position and attached to the inner tube in accordance with the present disclosure.

FIG. 20 shows a simplified diagram of a male urinary tract system in which a catheter may be inserted in accordance with the present disclosure.

FIG. 21 shows a catheter having an inflatable retaining balloon in accordance with the present disclosure.

FIG. 22 shows a catheter assembly connected to a urinary bag in accordance with the present disclosure.

FIG. 23 shows a catheter assembly comprising an inner tube and an outer tube in accordance with the present disclosure.

FIG. 24 shows a catheter assembly comprising an inner tube and an outer tube in accordance with the present disclosure.

FIG. 25 shows a catheter assembly comprising an inner tube and an outer tube in accordance with the present disclosure.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, ingredients, steps, etc. are optionally present. For example, an article “comprising” components A, B, and C can contain only components A, B, and C, or can contain not only components A, B, and C, but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

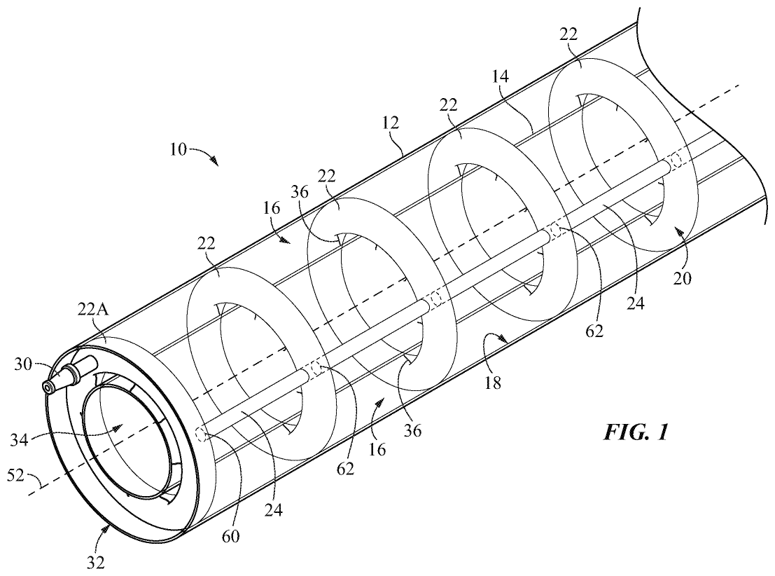

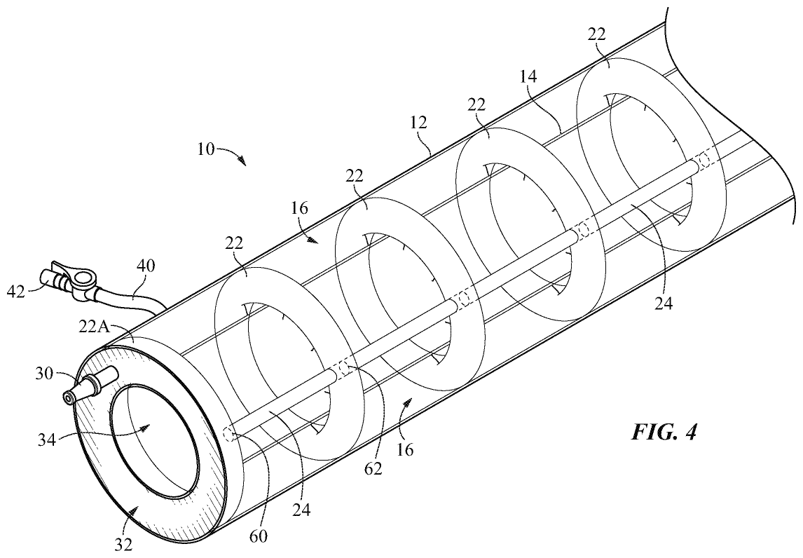

In one aspect, a catheter 10 having an inflatable and deflatable expansion tube structure 20 for occlusion and dilation of a lumen 34 of the catheter 10 and a method of using the catheter 10 are provided. FIGS. 1-3 illustrate one preferred embodiment of the catheter 10, and FIGS. 4-19 illustrate various alternative embodiments of the catheter 10. The catheter 10 generally comprises a flexible outer tube 12, the expansion tube structure 20 disposed within the outer tube 12, and, optionally, a flexible inner tube 14 also disposed within the outer tube 12. Each of these components is made of a flexible material that allows the components to bend without breaking. The inflatable expansion tube structure 20 allows the catheter 10 to expand and contract to transition the catheter 10 between an inflated configuration and a deflated configuration, respectively. When the catheter 10 is in the inflated configuration, the lumen 34 of the catheter 10 is expanded to its maximum diameter to allow the maximum flow of fluid through the lumen 34 or to allow intraluminal passage of instrumentation into an organ system that dilates and constricts either pathologically or physiologically. When the catheter 10 is in the deflated configuration, the lumen 34 is substantially occluded to restrict the flow of fluids through the lumen 34. In some embodiments, the lumen 34 may be partially closed to restrict flow, and the expansion tube structure 20 may be partially inflated to control the size of the lumen 34 and thus control the flow of fluid through the lumen 34. FIGS. 3A, 6A, 18, and 19 show embodiments of the catheter 10 with the expansion tube structure 20 in the deflated configuration, and the remaining figures show the catheter 10 with the expansion tube structure 20 in the fully inflated configuration to illustrate the fully expanded shape of each of the flexible components of the catheter 10. When in the deflated configuration, all or substantially all fluids may be removed from all components of the catheter 10, thereby causing all flexible components of the catheter 10 to substantially or entirely collapse to minimize the size of the catheter 10 as much as possible. The expansion tube structure 20 may also be partially inflated to varying degrees to control the size of the lumen 34.

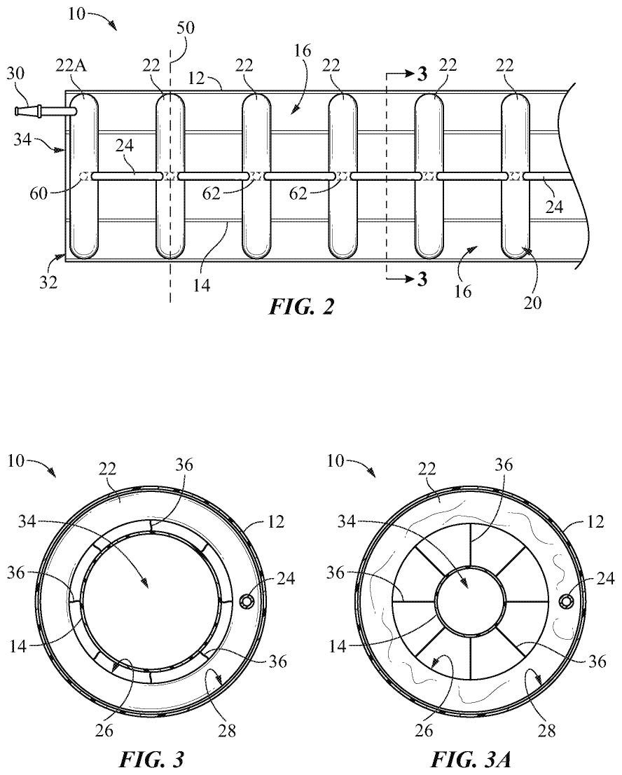

The expansion tube structure 20 is attached to or embedded within an interior wall 18 of the flexible outer tube 12. In a preferred embodiment, as shown in FIGS. 1-10 and 16-19, the expansion tube structure 20 comprises a plurality of individual tubular rings 22. The tubular rings 22 are arranged in series and disposed in spaced relation along a length of the outer tube 12, as best seen in FIGS. 1 and 2. Each of the individual tubular rings 22 defines an annular fluid chamber that may be filled with fluid to expand the annular fluid chamber and transition the catheter 10 into the inflated configuration. The expansion tube structure 20 further comprises one or more inflation tubes 24 fluidly connecting each tubular ring 22 to an adjacent tubular ring 22 such that the annular fluid chambers of all of the tubular rings 22 are fluidly connected to each other to define a single fluid chamber extending throughout each of the plurality of tubular rings 22 and the one or more inflation tubes 24 of the catheter 10. The one or more inflation tubes 24 may comprise a plurality of individual lengths of tube 24 with each respective length of tube 24 extending directly between two adjacent tubular rings 22 of the plurality of tubular rings 22. In other embodiments, the one or more inflation tubes 24 may comprise a single tube structure that is fluidly connected to each of the tubular rings 22.

The plurality of tubular rings 22 may preferably comprise an end tubular ring 22A disposed at each of two opposing ends 32 of the catheter 10 (one of which is shown in FIGS. 1 and 2) and a plurality of middle tubular rings 22 disposed between the two opposing end tubular rings 22A. Thus, the inflation tube 24 may extend from only one side of the end tubular ring 22A to the adjacent tubular ring 22, and the inflation tube 24 may extend from each of two opposing sides of each of the middle tubular rings 22 to two opposing adjacent tubular rings 22. In a preferred embodiment, as best seen in FIGS. 1 and 2, the inflation tube 24 may comprise a single relatively rigid tube, which may preferably be more rigid than the rings 22, that extends into each of the tubular rings 22 and through each of the middle tubular rings 22. The inflation tube 24 may have an opening 62 in the tube structure to allow controlled flow of an inflation fluid into each of the individual rings 22 along the length of the inflation tube 24, which may allow each of the plurality of tubular rings 22 to simultaneously fill more evenly during the process of inflating the expansion tube structure 20 to transition the catheter 10 to the inflated configuration. The diameter of each of the openings 62 may be smaller than a width of each of the tubular rings 22. When the catheter 10 is in a partially inflated configuration, the openings 60 may allow each of the plurality of tubular rings 22 to more quickly reach an equilibrium of approximately equal partial inflation between each of the individual rings 22. The inflation tube 24 may extend into the end tubular ring 22A so that an open end 60 of the inflation tube 24 is fluidly connected to the annular fluid chamber of the end tubular ring 22A.

The catheter 10 further comprises an injection port 30 connected to one of the tubular rings 22A that is disposed at one end 32 of the outer tube 12. The injection port 30 is configured to allow injection of the inflation fluid into the annular fluid chambers of all of the tubular rings 22 and one or more connecting inflation tubes 24 through the injection port 30 to transition the catheter 10 into the inflated configuration. The inflation fluid is used to substantially fill the entire single fluid chamber that is defined by all of the tubular rings 22 and connecting inflation tube 24 to transition the catheter 10 into the inflated configuration or into a configuration of any desired degree of partial inflation. When inflation fluid is injected into the tubular rings 22 and connecting inflation tube 24 via the injection port 30 to transition the catheter 10 into the inflated configuration, each of the tubular rings 22 expands radially outward relative to a longitudinal axis 52 of the catheter 10 to expand the diameter of the lumen 34. The inflation fluid is preferably a sterile saline solution or similar sterile liquid. Alternatively, the inflation fluid may be a gas. The injection port 30 is further configured to allow withdrawal of the inflation fluid from the annular fluid chambers of all of the tubular rings 22 through the injection port 30 to transition the catheter 10 into the deflated configuration. As best seen in FIG. 1, the injection port 30 may comprise a nozzle having an open end that is configured to allow a separate fluid supply line (not shown) to be attached to the injection port 30 to supply the inflation fluid to the expansion tube structure 20 and to evacuate the inflation fluid from the expansion tube structure 20. The fluid supply line may have a valve to control the flow of the inflation fluid into and out of the expansion tube structure 20.

As best seen in FIG. 2, each of the plurality of tubular rings 22 is preferably disposed in a position that is generally transverse to the lumen 34 when the catheter 10 is in the inflated configuration. Further, each tubular ring 22 may define a plane 50 that dissects the annular fluid chamber of each tubular ring 22 into two annular rings. In a preferred embodiment, this dissecting plane 50 is generally perpendicular to an axis 52 that is defined by a flow path of the lumen 34 when the flexible catheter 10 is positioned along a generally straight line in the inflated configuration. In a preferred embodiment, the plurality of tubular rings 22 are equidistantly spaced along the length of the catheter 10. In addition, each individual tubular ring 22 is attached to or embedded within the interior wall 16 of the outer tube 12, preferably around an entire perimeter 28 of each tubular ring 22. Thus, there is preferably a continuous fluid-tight attachment between the interior wall 18 of the outer tube 12 and each tubular ring 22 around the outer perimeter 28 of each tubular ring 22 such that fluid cannot flow between the interior wall 18 of the outer tube 12 and any of the tubular rings 22. The transverse position of each tubular ring 22 and continuous attachment point of each tubular ring 22 to the interior wall 18 of the outer tube 12 may allow for maximum expansion of the outer tube 12 and thus maximum expansion of the lumen 34 when the tubular rings 22 are filled with inflation fluid to transition the catheter 10 into the inflated configuration.

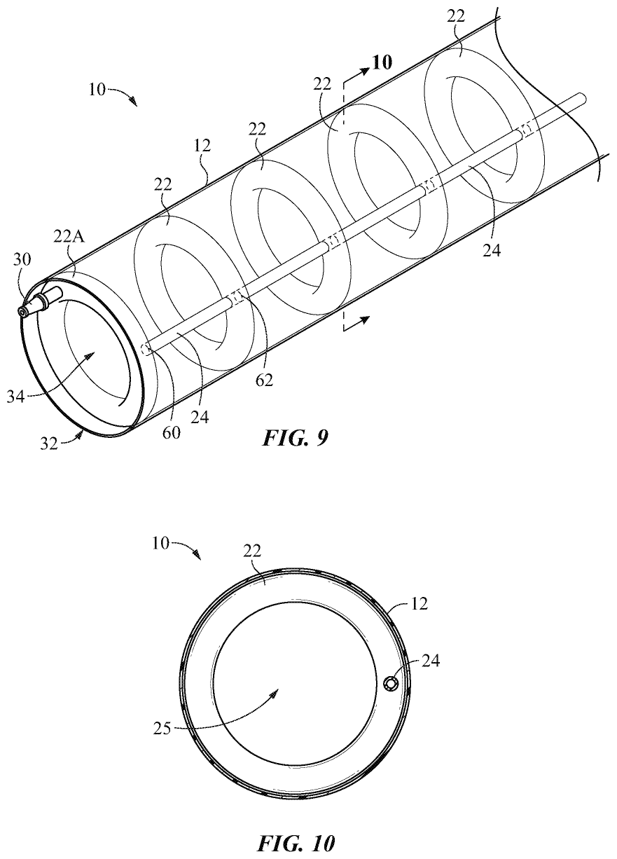

In one embodiment, as shown in FIGS. 9 and 10, the lumen 34 of the catheter 10 may be defined by the outer tube 12 and by each of the plurality of tubular rings 22, each of which has a central 25 opening that may in part define the lumen 34. In a preferred embodiment, as shown in FIGS. 1-8, the catheter 10 further comprises a flexible inner tube 14 that is attached to each of the plurality of tubular rings 22. The inner tube 14 extends through the central opening 25 of each of the plurality of tubular rings 22. In this embodiment, the lumen 34 may be defined by the inner tube 14, which may allow the occluded lumen 34 to more effectively restrict the flow of fluids through the lumen 34 when the catheter 10 is in the deflated configuration. The inner tube 14 may also allow more effective control of the flow of fluids through the lumen 34 when the catheter 10 is in any state of partial inflation. When inflation fluid is evacuated from the expansion tube structure 20 to transition the catheter 10 from the inflated configuration to the deflated configuration, the tubular rings 22 and outer tube 12 may contract and partially collapse, thereby causing the inner tube 14 to also contract and collapse. With the inner lumen 34 that provides for fluid flow through the catheter 10 being defined by the inner tube 14, closure of the inner tube 14 may more effectively occlude the lumen 34 and thus may more effectively restrict the flow of fluids through the lumen 34. In this embodiment, the inner lumen 34 is defined by a single structure, which is the inner tube 14, which may provide a more restrictive closure of the lumen 34 along the entire length of the inner tube 14. This characteristic may be beneficial in certain applications of the catheter 10, such as use of the catheter 10 as a urinary tract catheter to drain urine from a patient's bladder, in which case leakage of urine from the catheter 10 is undesirable when the catheter 10 is left in place but not being used for draining urine.

As best seen in FIGS. 1 and 3, the inner tube 14 is preferably attached to each of the plurality of tubular rings 22 by a plurality of short filaments 36 or tethers. The filaments 36 are preferably made of polypropylene or a similar flexible material. Each filament 36 may be attached to an inner perimeter 26 of one of the tubular rings 22 and to an outer surface of the inner tube 14. In this embodiment, the outer tube 12 and the inner tube 14 define an annular space 16 disposed between the outer tube 12 and the inner tube 14. The volume of the annular space 16 may be maximized when the inner tube 14 is occluded, and the volume of the annular space 16 may be minimized when the inner tube 14 is fully dilated. FIG. 3A shows the catheter 10 in a generally deflated state. In this instance, the lumen 34 is not fully occluded but is only partially closed, which may allow some restriction of the lumen 34 to control flow through the lumen 34. The filaments 36 may stretch or contract depending on the physical state of the catheter 10, which may affect the distance between the inner tube 14 and the outer tube 12. The attachment of the inner tube 14 to the tubular rings 22 by filaments 36 allows fluid to flow around the filaments 36 between the exterior of the inner tube 14 and the interior perimeter 26 of the tubular rings 22. Thus, attachment of the inner tube 14 to the tubular rings 22 by filaments 36 allows any fluid that may be contained within the annular space 16 to flow along the entire length of the catheter 10 without becoming trapped within portions of the annular space 16 disposed between any adjacent tubular rings 22.

For instance, when the catheter 10 is transitioned from the inflated configuration to the deflated configuration, the volume of the annular space 16 may expand, which may draw air outside the catheter 10 into the annular space 16 through an open end 32 of the catheter 10. This air may then be evacuated through the same open end 32 when the catheter 10 is transitioned back to the inflated configuration after use. In this embodiment, as best seen in FIG. 1, the end 32 of the catheter 10 may be open between the outer tube 12 and the inner tube 14. In an alternative embodiment, as shown in FIG. 4, the end 32 of the catheter 10 may be closed between the outer tube 12 and the inner tube 14. In this embodiment, the catheter 10 may further comprise an equalization port 40 that is fluidly connected to the annular space 16, preferably through an opening in the sidewall of the outer tube 12 at a location near the end 32 of the catheter 10, to equalize pressure between the annular space 16 and the exterior of the catheter 10. The equalization port 40 may preferably include a valve 42 configured to open and close to allow and restrict flow through the valve 42. The valve 42 may be opened to equalize pressure across the valve 42 and closed to maintain pressure within the annular space 16. In this embodiment, a fluid supply line (not shown) may optionally be connected to the equalization port 40 to supply a fluid into the annular space 16, such as a sterile saline solution or other suitable fluid to at least partially fill the annular space 16 when the catheter 10 is in the inflated configuration. When the annular space 16 is sufficiently filled with fluid, the valve 42 may be closed to maintain the pressure within the annular space 16. The valve 42 may then be opened to equalize the pressure and evacuate fluid from the annular space 16 when the catheter 10 is transitioned back to the deflated configuration.

FIG. 4 shows a proximal end 32 of the catheter 10 that remains outside of the body of the patient during use to provide access to the injection port 30 and optional equalization port 40. In a preferred embodiment, an opposing distal end (not shown) of the catheter 10 may also be closed between the outer tube 12 and the inner tube 14 so that bodily fluids of the patient cannot enter the annular space 16 through the distal end of the catheter 10, which ensures that bodily fluids can only flow through the lumen 34 that is defined by the inner tube 14.

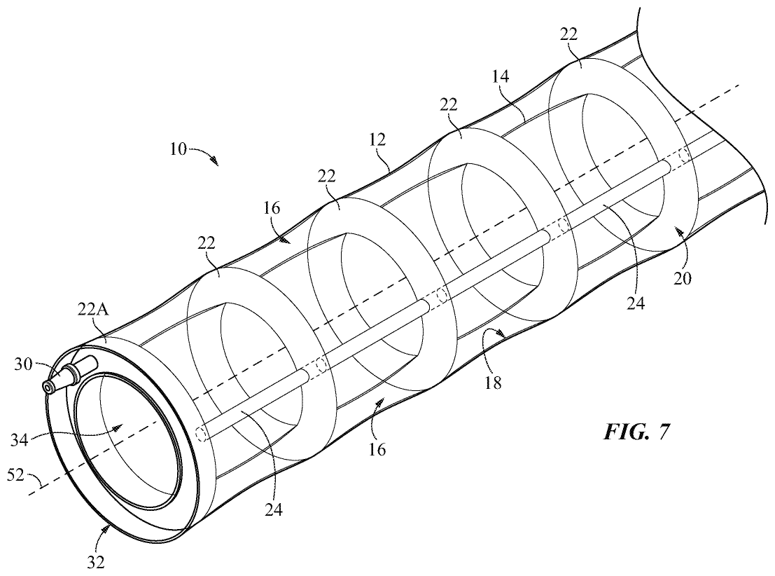

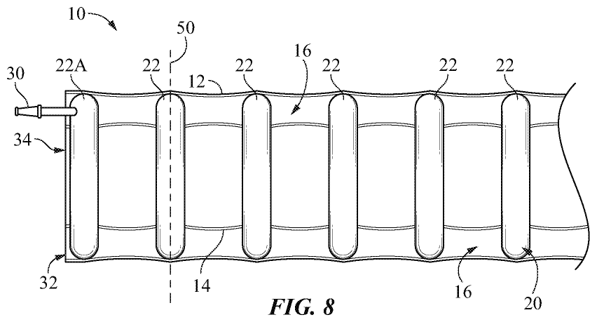

In one embodiment, as shown in FIGS. 7 and 8, the inner tube 14 may be adhered to the tubular rings 22 to form a fluid tight attachment between the inner tube 14 and the tubular rings 22 so that fluid cannot flow between the exterior of the inner tube 14 and the interior perimeter 26 of the tubular rings 22. In this case, the annular space 16 may define isolated pockets between the inner tube 14 and the outer tube 12 and between each pair of adjacent tubular rings 22 in the series of rings 22. These pockets may have some amount of fluid, such as a sterile saline solution, contained within the pockets. When the tubular rings 22 are inflated, a partial vacuum may form in the annular space 16 due to the isolated pockets of fluid. In this case, portions of the wall of the inner tube 14 and the wall of the outer tube 12 between tubular rings 22 may biased inwardly toward each other. This embodiment may be utilized when the catheter 10 is used to control flow through the lumen 34 without requiring full occlusion of the lumen 34.

The outer tube 12, the inner tube 14, and the inflatable expansion tube structure 20 are each made of a flexible material, which is preferably a rubber or plastic polymer or copolymer material, such as polypropylene, that is suitable for internal use inside the body of a human patient. In a preferred embodiment, the inner tube 14 and the inflatable expansion tube structure 20 are both made of a highly pliable material that allows the inner tube 14 and the expansion tube structure 20 to fully contract and collapse to a minimum size when the catheter 10 is in the deflated configuration to maximize the extent to which fluid flow is restricted in the occluded lumen 34. In a preferred embodiment, the outer tube 12 may be made of a generally flexible material that will contract to some extent when the catheter 10 is in the deflated configuration but that retains a relative level of rigidity that allows the catheter 10 to be inserted into the body of the patient. Alternatively, the catheter 10 may optionally include an additional structural component (not shown) to facilitate insertion. For instance, a sheath that is flexible but more rigid than the outer tube 12 may be disposed around the exterior of the outer tube 12 and used to guide the distal end of the catheter 10 to a desired location within the body of the patient. The sheath may then be withdrawn, thereby leaving the catheter 10 in place. Alternatively, a semi-rigid rod (not shown) may be inserted through the inner tube 14 and removably secured to the distal end of the catheter 10. The catheter 10 with the semi-rigid rod may then be inserted to the desired location, and the rod may then be detached from the distal end of the catheter 10 and withdrawn, thereby leaving the catheter 10 in place within the body of the patient. In different embodiments, the outer tube 12 may have varying degrees of flexibility. For instance, in some embodiments, the outer tube 12 may be more rigid so that the outer tube 12 does not substantially change shape when the catheter 10 is transitioned to the deflated configuration, and in other embodiments the outer tube 12 may be more flexible and/or pliable so that the outer tube 12 substantially collapses when the catheter 10 is transitioned to the deflated configuration. The degree of flexibility and/or pliability of the outer tube 12 may depend on the particular application and the degree to which occlusion of the lumen 34 is desired.

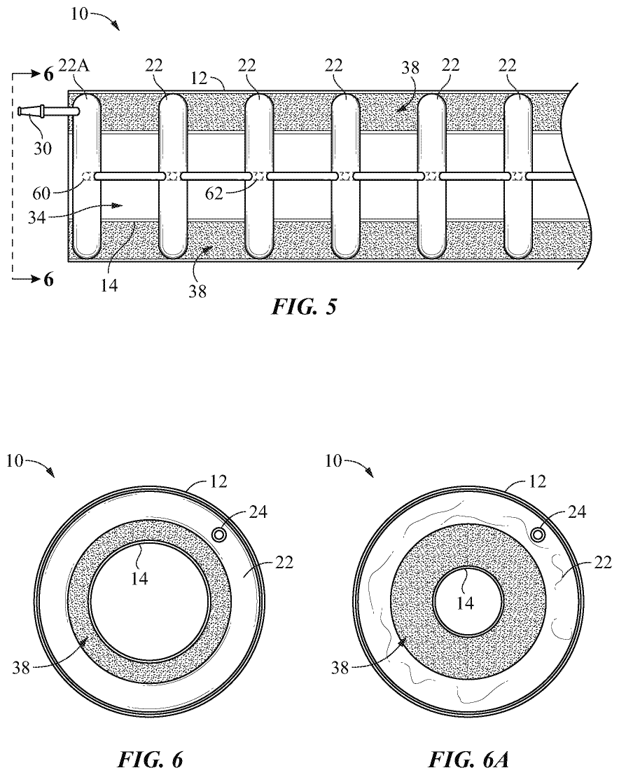

In an alternative embodiment, as shown in FIGS. 5, 6, and 6A, an expandable rubber material 38 may be disposed within the annular space 16 such that the expandable rubber material 38 substantially fills the annular space 16 when the catheter 10 is in either the inflated or deflated configuration. In this embodiment, the rubber material 38 may naturally contract when the catheter 10 is in the deflated configuration and then expand when the catheter 10 transitions into the inflated configuration. In this embodiment, the process of injecting fluid into the annular fluid chambers of the tubular rings 22 to transition the catheter 10 into the inflated configuration causes the outer tube 12 to fully expand to a maximum diameter, which forces the rubber material 38 to expand, thereby causing the lumen 34 to dilate. FIGS. 5 and 6 show the catheter 10 in the inflated configuration, and FIG. 6A shows the catheter 10 in the deflated configuration. In this embodiment, the catheter 10 may include the inner tube 14, which may be adhered to or otherwise attached to the rubber material 38, in which case the lumen 34 is defined by the inner tube 14. Alternatively, the lumen 34 may be defined by a central longitudinal conduit formed by the rubber material 38 itself. As best seen in FIG. 6, the diameter of the lumen 34 when the catheter 10 is in the inflated configuration is preferably smaller than the diameter of the central opening 25 of each of the tubular rings 22. As shown in FIG. 6A, the diameter of the lumen 34 when the catheter 10 is in the inflated configuration may not be fully occluded and may thus be used in applications in which it is desired to control the flow through the lumen 34 without requiring full occlusion of the lumen 34.

The embodiment shown in FIGS. 9 and 10, does not include an inner tube 14. Thus, the lumen 34 in this embodiment is defined by portions of the outer tube 12 and tubular rings 22. This embodiment may also be utilized primarily in applications in which it is desired to control the flow through the lumen 34 without requiring full occlusion of the lumen 34.

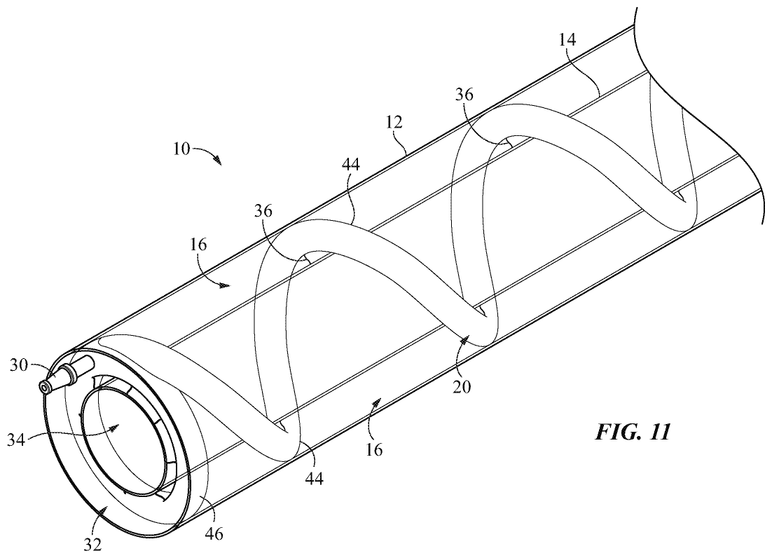

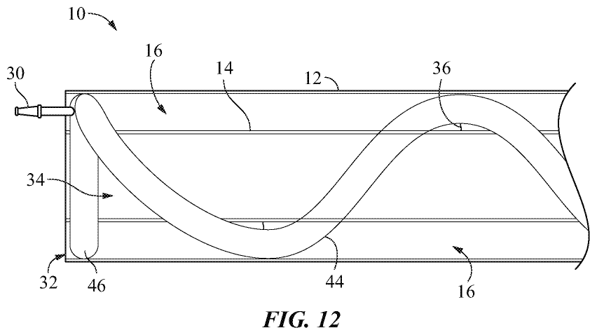

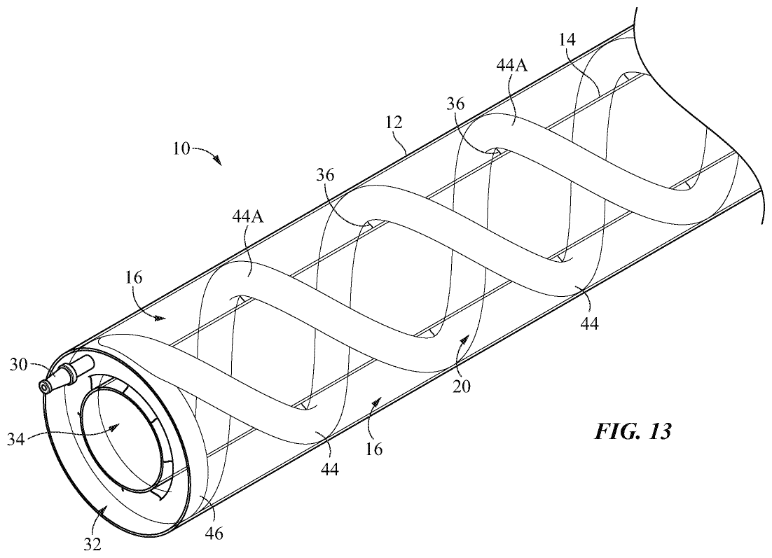

In other embodiments, as shown in FIGS. 11-15, the expansion tube structure 20 comprises at least one helical inflation tube 44 attached to or embedded within the interior wall 18 of the outer tube 12 in a helical configuration to define a helical fluid chamber that extends through the entire length of the helical inflation tube 44. As shown in FIGS. 11 and 12, the expansion tube structure 20 may comprise a single helical inflation tube 44. Alternatively, as shown in FIGS. 13-15, the expansion tube structure 20 may comprise a first helical inflation tube 44 and a second helical inflation tube 44A that form a double helix.

The injection port 30 is connected to helical inflation tube 44 and/or 44A at an end 32 of the catheter 10 to facilitate transitioning the catheter 10 between the inflated and deflated configurations. In a preferred embodiment, the expansion tube structure 20 further comprises a tubular ring 46 that is fluidly connected to helical inflation tube 44 and/or 44A at an end of the helical inflation tube 44, which is generally positioned at the proximal end 32 of the outer tube 12. The tubular ring 46 provides structural support to the proximal end 32 of the outer tube 12 to maintain the open end of the lumen 34 in a state of full expansion when the catheter 10 is in the inflated configuration. In this embodiment, the injection port 30 is preferably connected to the tubular ring 46, as best seen in FIG. 11. When fluid is injected into the helical fluid chamber via the injection port 30 to transition the catheter 10 into the inflated configuration, helical inflation tube 44 and/or 44A expands radially outward relative to axis 52 to expand the diameter of the lumen 34, which may be defined by the outer tube 12 and helical inflation tube 44 and/or 44A. When fluid is withdrawn from the helical fluid chamber of helical inflation tube 44 and/or 44A to transition the catheter 10 into the deflated configuration, helical inflation tube 44 and/or 44A contracts inwardly to constrict the diameter of the lumen 34 to restrict the flow of fluid through the lumen 34. In this embodiment, the catheter 10 may optionally include a flexible inner tube 14, which is preferably attached to helical inflation tube 44 and/or 44A by a plurality of short filaments 36 or tethers disposed along the length of the inner tube 14, as best seen in FIGS. 11 and 12. In this case, the lumen 34 may be defined by the inner tube 14.

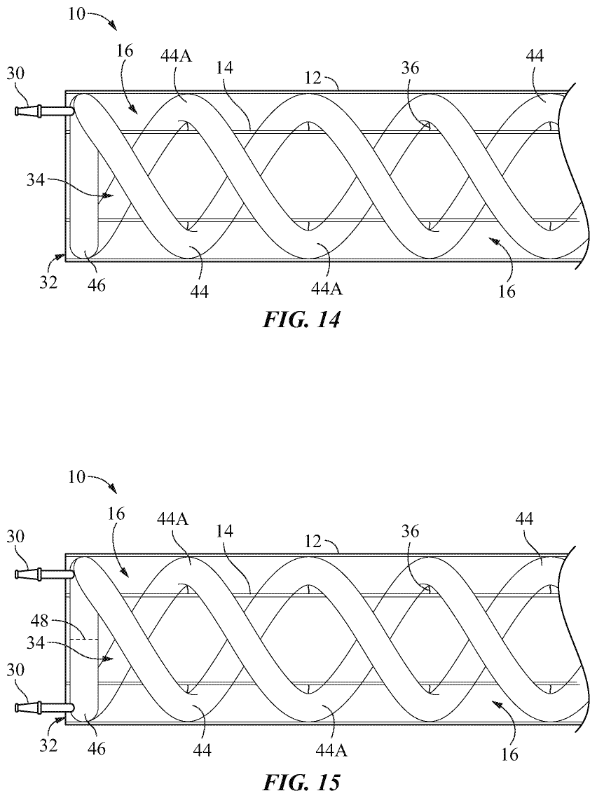

In one embodiment, the expansion tube structure 20 may comprise a single injection port 30 that is fluidly connected to both the first helical inflation tube 44 and the second helical inflation tube 44A so that inflation fluid can be simultaneously supplied to or evacuated from both helical inflation tubes 44 and 44A, as shown in FIGS. 13 and 14. In one preferred embodiment, as shown in FIG. 15, the expansion tube structure 20 may comprise two separate injection ports 30 that are fluidly connected to each respective one of the first helical inflation tube 44 and the second helical inflation tube 44A so that inflation fluid can be independently supplied to or evacuated from each of the helical inflation tubes 44 and 44A. In a preferred embodiment, helical inflation tubes 44 and 44A may both be fluidly connected to a single tubular ring 46, which may have an internal divider wall 48 that isolates the fluid chambers of helical inflation tubes 44 and 44A from each other. In the embodiment of the catheter 10 utilizing a double helix with two injection ports 30, the process of injecting inflation fluid into each of the two separate fluid chambers that are defined by helical inflation tubes 44 and 44A may be utilized to steer a distal end of the catheter 10 into or within an organ of the body of a patient. For instance, injecting inflation fluid into only tube 44 may cause the distal end of the catheter 10 to turn in one direction, and then injecting inflation fluid into only tube 44A while holding the fluid in tube 44 may cause the distal end of the catheter 10 to turn in an opposing direction. Similarly, evacuating fluid from only one of the inflation tubes 44 or 44A may also cause the catheter 10 to move in a defined direction. Thus, in one preferred embodiment, inflation fluid may be independently injected, held to maintain pressure, and/or evacuated from each of the two separate tubes 44 and 44A to perform complex maneuvers to position the distal end of the catheter 10 in a desired position within the body of the patient.

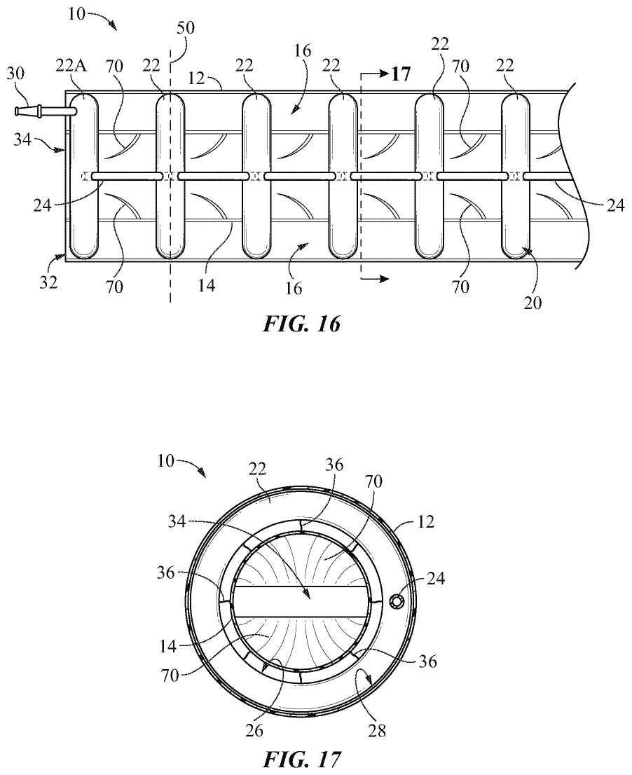

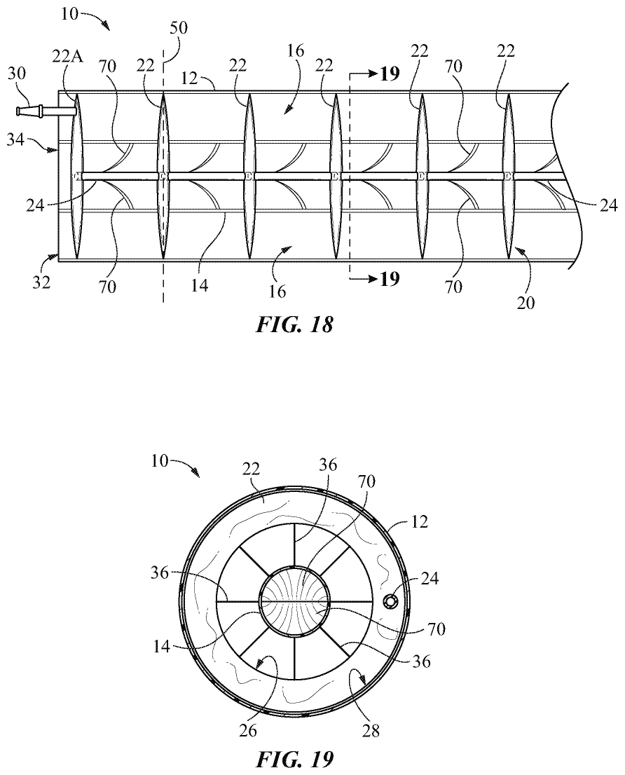

In another embodiment, as shown in FIGS. 16-19, the catheter 10 comprises a plurality of valves each comprising a pair of opposing flexible flaps 70. Each of the valve flaps 70 is attached to an interior wall of the inner tube 14 and disposed in spaced relation along a length of the inner tube 14. Alternatively, the valve flaps 70 may be attached to the tubular rings 22 or to the outer tube 12 in embodiments of the catheter that do not include the inner tube 14. The inner tube 14 and each of the plurality of tubular rings 22 define the lumen 34 when the catheter 10 is in the inflated configuration. The two opposing flaps 70 of each valve are configured to substantially close the lumen 34 to fluid flow through the lumen 34 when the catheter 10 is in the deflated configuration, as shown in FIGS. 18 and 19, and the two opposing flaps 70 of each valve are configured to separate and open the lumen 34 to fluid flow through the lumen 34 when the catheter 10 is in the inflated configuration, as shown in FIGS. 16 and 17. Thus, inflation of the tubular rings 22 draws the opposing flaps 70 of each valve apart from each other to open the lumen 34, and deflation of the tubular rings 22 allows the valve flaps 70 to move back toward each other and contact each other to substantially close the lumen 34.

In a preferred embodiment, each of the valve flaps 70 is attached to the inner tube 14 at a proximal or base end of the flap 70 and extends away from the inner tube 14 toward a distal end of the flap 70 in a curved orientation to form a generally curved structure extending between the proximal end and distal end of each flap 70. As best seen in FIG. 16, the flap 70 of each valve curves in the same direction, which, in this case, is toward the proximal end of the catheter 10 at which the injection port 30 is located. The curved shape of each flap 70 allows the flaps 70 to more effectively close the lumen 34 since a larger surface area of each flap 70 may contact the opposing flap 70 when the valve is closed, as best illustrated in FIG. 19. The curved shape of each flap 70 also allows each valve formed by two opposing flaps 70 to function as a one-way valve. For instance, the orientation of the valve flaps 70 as shown in FIG. 16 allows fluid to flow only toward the proximal end of the catheter (right-to-left in the perspective shown in FIG. 16) even when the lumen 34 is open. If any fluid begins to flow in the opposite direction (toward the distal end of the catheter), flow in this direction will cause each of the flaps 70 to move toward its corresponding opposing flap 70, which will cause the valve to close and prevent the flow of fluids in that direction. This feature may be advantageous in some medical applications. For instance, when used in the urinary tract, the valve flaps 70 may allow urine to flow from the patient's bladder to the proximal end of the catheter 10 but will prevent backflow of fluid into the patient's bladder. In an alternative embodiment, the valve flaps 70 may curve toward the distal end of the catheter 10.

A method of using the catheter 10 is also provided. First, the catheter 10 may be inserted into an organ of a human patient, at which time the catheter 10 may be in the deflated configuration, the inflated configuration, or in a semi-inflated configuration in which some inflation fluid has been injected into the expansion tube structure 20 to partially fill the single fluid chamber that is defined by either helical inflation tube 44 and/or 44A or the plurality of tubular rings 22 with connecting inflation tubes 24. The catheter 10 may be inserted independently or, optionally, using a more rigid sheath or rod to aid insertion. The catheter 10 may then be transitioned to the fully inflated configuration to fully expand the lumen 34 to its maximum diameter, which allows fluid flow or instrument insertion through the lumen 34, or to the fully deflated configuration, which substantially occludes the lumen 34 and thus substantially restricts the flow of fluids through the lumen 34, or to a partially inflated configuration, which may be utilized to control the flow of fluids through the lumen 34 to a desired rate of flow. Inflation fluid may be injected into the expansion tube structure 20 or evacuated from the expansion tube structure 20 through the injection port 30. The optional equalization port 40 may be utilized to equalize pressure between the annular space 16 and the environment external to the catheter 10 in embodiments that utilize an inner tube 14 and that have a closed proximal end 32.

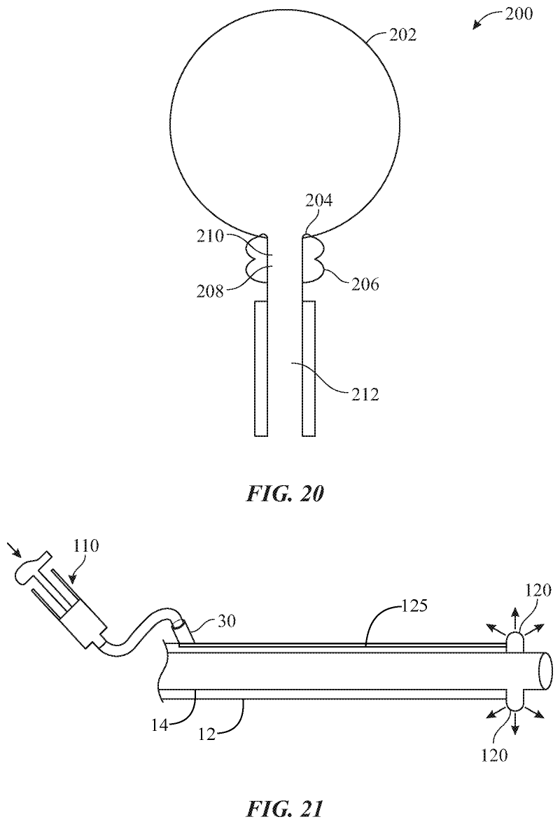

In a preferred embodiment, the catheter 10 is used to control the flow of urine through the urinary tract 200, visualized in FIG. 20. The catheter 10 is preferably designed to occupy the lumen of the segments of the urethra, from the penile urethra 212 through the membranous urethra 208 to the prostate urethra 210. In another preferred embodiment, the distal end of the catheter 10 is localized to the bladder 202. When in a deflated state, the catheter 10 is held closed to prevent the flow of urine by the internal urethral sphincter 204 and the external urethral sphincter 206. This transient, reversable occlusion provides a healthcare provider direct control over the flow of urine and reduces the risk of urine backflow and urinary tract infection.

FIG. 21 illustrates an embodiment of the catheter further comprising a retaining balloon 120. The function of the retaining balloon 120 is to anchor the catheter 10 in place. In one preferred embodiment, the retaining ballon 120 forms an annular ring about the distal end of the catheter 10. In another preferred embodiment, the retaining balloon 120 is localized to only one side of the catheter 10 and does not form a ring structure. In still another preferred embodiment, the retaining ballon 120 is substantially spherical. In yet another preferred embodiment, the retaining balloon 120 is composed of a material which is more elastic than the rest of the catheter 120. For instance, the retaining balloon 120 might be composed of silicone rubber.

In a preferred embodiment, the retaining balloon 120 is inflated with a balloon fluid once the distal end of the catheter 10 reaches the bladder 202, thereby preventing the accidental dislodging or removal of the catheter 10. The balloon fluid is preferably saline, particularly sterile saline to reduce the risks of infection to the patient if the retaining balloon 120 leaks or ruptures. In another preferred embodiment, the retaining balloon 120 is inflated using a syringe 110 and injection port 30 linked to the retaining balloon 120 by a linking tube 125. In yet another preferred embodiment, the linking tube is flexible to accommodate the necessary turns and bend of the catheter 10, but it is not expandable. These characteristics prevent it from interfering with the necessary inflation and deflation of the catheter 10. It is recommended that the linking tube 125 be fluidly isolated from any expansion tube structure or the tubular fluid chamber 38 so that the retaining balloon 120 may remain inflated or deflated regardless of the inflation or deflation of the catheter 10 overall.

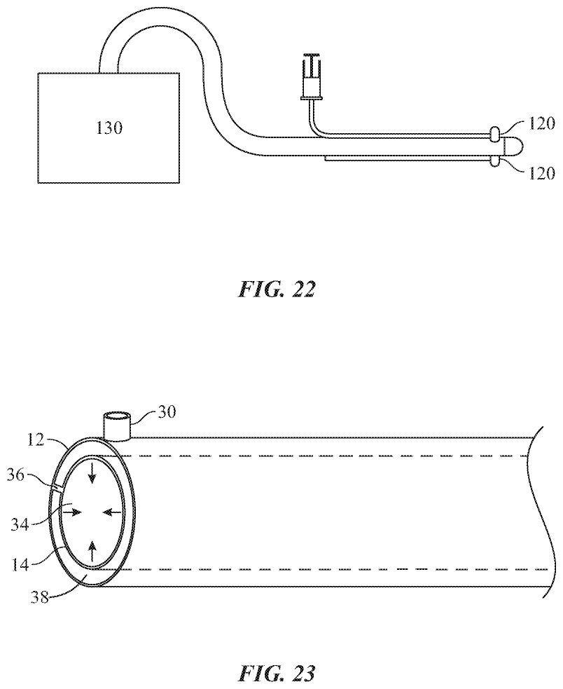

FIG. 22 illustrates a catheter assembly coupled to a collection bag 130 suitable for collection of bodily fluids. In a preferred embodiment, the collection bag 130 is a urinary drainage bag reversibly coupled to the proximal end of the catheter 10. In another preferred embodiment, the collection bag 130 is used to store kidney stones, gallstones, blood clots, and other non-fluid bodily products. It is recommended that the interior of the collection bag 130 be sterile to further reduce the risk of backflow and infection. The length of the catheter 10 should be calibrated both to accommodate sex-based differences in the urethra and to ensure that the catheter 10 can reach a mounted bag as is commonly found in clinical settings. In yet another preferred embodiment, only the sections of the catheter 10 projected to be inside the urethra have the inflatable interior, with the remainder left an ordinary tube.

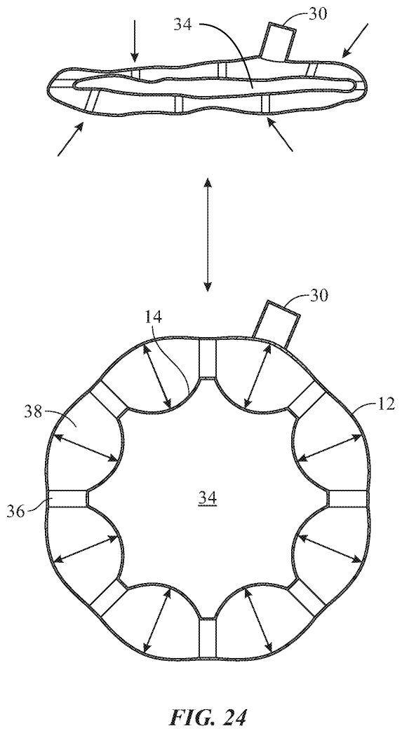

FIG. 23 shows an embodiment of the catheter 10 lacking an expansion ring structure. In this embodiment, the catheter 10 comprises an outer tube 12 and an inner tube 14. The outer tube 12 and inner tube 14 define both a lumen 34 and a tubular fluid chamber 38 localized between and defined by the outer tube 12 and the inner tube 14. For optimal function, the outer tube 12 may have a differing elasticity to the inner tube 14. Fluid is added to the tubular fluid chamber 38 by means of the injection port 30 to open or occlude the lumen 34. In a preferred embodiment, the fluid is a sterile saline solution.

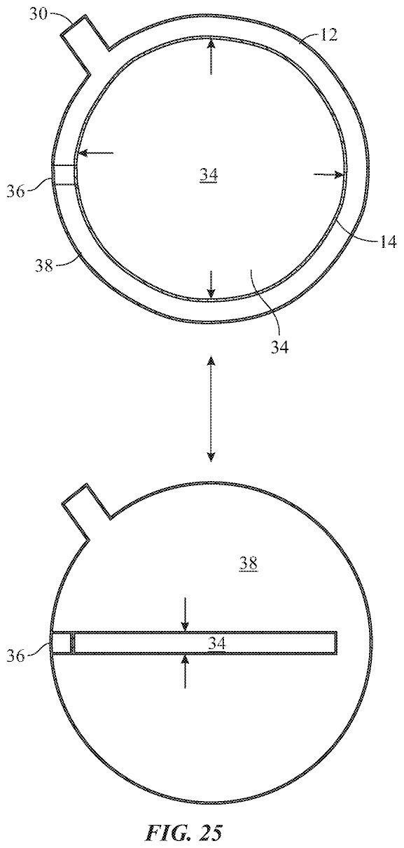

FIGS. 24-25 illustrate two embodiments of a catheter 10 lacking an expansion ring structure. In a preferred embodiment, the catheter 10 further comprises one or more filaments 36 linking the inner tube 14 to the outer tube 12. The number and spacing of the filaments 36 affects the mechanism by which fluid in the tubular fluid chamber 38 opens or occludes the lumen 34. FIG. 24 illustrates an embodiment of the catheter 10 having multiple filaments 36 substantially evenly spaced around the inner tube 14. In this preferred embodiment, the inner tube 14 and the outer tube 12 are pliable and easily compressed by the urethra while deflated. When the catheter 10 is compressed, the lumen 34 is substantially occluded. However, when fluid is added to the tubular fluid chamber 38 via the injection port 30, the fluid exerts pressure upon the outer tube 12 and the inner tube 14. This pressure transitions the outer tube 12 into a generally cylindrical shape capable of resisting the compressive forces exerted by the urethra.

FIG. 25, by contrast, illustrates an embodiment of the invention having few, singular, or unevenly dispersed filaments 36 affixing the inner tube 14 to the outer tube 12. In a preferred embodiment, the outer tube 12 is capable of resisting compression by the urethra, thereby enabling the catheter 10 to maintain a substantially defined inner volume comprising an inner tube 14, lumen 34, filaments 36, and tubular fluid chamber 38. In such an embodiment with a defined total volume, the addition of fluid to the tubular fluid chamber 38 applies pressure to the exterior of the inner tube 14, causing it to compress and occlude the lumen 34. When fluid is withdrawn, the tubular fluid chamber will contract, withdrawing the inner tube 14 from the lumen 34 and enabling the flow of bodily fluids therethrough. The embodiments of the catheter 10 depicted in FIGS. 24-25 have been illustrated with their lumens 34 substantially occluded, not completely occluded. However, one with skill in the art will recognize that the lumens 34 may be entirely occluded using the aforementioned designs and that the substantial occlusion depicted herein is for illustrative purposes only.

The present catheter 10 has numerous medical applications, particularly applications in which it is desired to maximize the diameter of the lumen 34 while continuously maintaining an open flow path for bodily fluids within an organ of the patient and/or applications in which it is desired to fully restrict the flow of bodily fluids through the lumen 34. For instance, the present catheter 10 may have applications for use in the urinary tract, the vasculature, the gastrointestinal system, and the pulmonary system. The catheter 10 may have additional uses, including, but not limited to, percutaneous uses and gynecological uses.

In some specific examples for use in the vascular system, the catheter 10 may be used to facilitate stent placement during simultaneous balloon angioplasty, in which case a stent can be passed intraluminal during inflation while allowing intraluminal blood flow during inflation and angioplasty. In other cases, the catheter can be used during balloon angioplasty in order to maintain the patency of an artery during inflation/dilation; it can be used to dilate vessels in order to pass instruments through the central lumen during intravascular surgery; it can be used in conjunction with a Fogarty catheter or other device to remove thrombus by pulling clot intraluminal and then closing the lumen to capture before removing; it can be used to tamponade a dissection and facilitate passage of stents or other instruments into area during procedure while maintaining hemostasis and intraluminal flow.

In some specific examples for use in the urinary tract, the catheter 10 may be used as a more advanced Foley catheter that opens and closes more effectively to allow and restrict flow through the lumen 34, which may prevent unwanted leakage of urine. The catheter can also be used to remove stones by pulling them intraluminal before closing and removing; it can be used to help pass stents in an area of ureter stricture; it can be used to open areas of stricture while maintaining patency and flow of lumen; and it can be used for dilation of urethra for instrumentation during procedure or stent placement.

In some specific examples for use in the gastrointestinal (GI) system, the catheter 10 may be used in the biliary system to facilitate stent placement, help retrieve stones and facilitate instrumentations through strictures or through the biliary tract for distal procedures; it can facilitate esophageal stenting or tamponade during variceal bleed; it can be utilized to mimic peristalsis in the GI system with wave like propagation/closing of the system; it can be used to balloon areas of stricture to pass instruments through anywhere within the GI tract; it can be used to facilitate pancreatic duct stenting, stone retrieval, instrumentation, temporary ballooning; it can be used as hemostatic tool during esophageal bleeds; and it can be used to remove specimens during lap or robotic surgery.

In some specific examples for use in the pulmonary system, the catheter 10 may be used in difficult airway situations to gain temporary airway, customize the diameter or allow for passing of different and more permanent airway through the inner lumen; it can allow tamponade for a bleeding pulmonary artery while allowing continued aeration of an effected lung segment; it can facilitate stent placement for strictures; it can facilitate bronchoscopy/stenting through intraluminal passing of instruments; and it can be used as temporizing measure during bleed into the upper airway.

In some specific examples for percutaneous uses, the catheter 10 may be used for dilation of tract during vascular line placement for larger catheter placement; it can be utilized to gain access to suprapubic catheter tracts that stricture; it can be utilized to dilate the tract of peg tubes that stricture; and it can be utilized to dilate tracts during drain placement into fluid collections.

In some specific examples for gynecological uses, the catheter 10 may be utilized to dilate the cervix to allow for instrumentation into the uterus; it can be utilized to dilate the vaginal canal for routine inspection in place of speculum for more comfort and customized diameter; and it can be utilized in other gynecological procedures in the uterus, cervix or fallopian tubes.

It will be appreciated that the configurations and methods shown and described herein are illustrative only, and that these specific examples are not to be considered in a limiting sense, because numerous variations are possible. The subject matter of the present disclosure includes all novel and non-obvious combinations and sub-combinations of the various systems and configurations, and other features, functions, and/or properties disclosed herein. It is understood that versions of the invention may come in different forms and embodiments. Additionally, it is understood that one of skill in the art would appreciate these various forms and embodiments as falling within the scope of the invention as disclosed herein.

What is claimed is:

1. A catheter comprising,
a flexible outer tube;
a helical inflation tube attached to an interior wall of said outer tube in a helical configuration to define a helical fluid chamber; and
an injection port connected to said helical inflation tube at an end of said catheter,
wherein said injection port is configured to allow injection of a fluid into said helical inflation tube through said injection port to transition said catheter into an inflated configuration, and wherein said injection port is further configured to allow withdrawal of said fluid from said helical inflation tube through said injection port to transition said catheter into a deflated configuration,
wherein said outer tube and said helical inflation tube define a lumen when said catheter is in said inflated configuration, and
wherein said lumen is substantially occluded when said catheter is in said deflated configuration.

2. The catheter of claim 1, further comprising a tubular ring fluidly connected to said helical inflation tube and disposed at an end of said helical inflation tube, wherein said injection port is connected to said tubular ring.

3. The catheter of claim 1, further comprising a flexible inner tube attached to an interior of said helical inflation tube by a plurality of filaments.

4. The catheter of claim 3, wherein said outer tube and said inner tube define a space disposed between said outer tube and said inner tube, wherein said catheter further comprises an expandable elastic material disposed within said space, wherein said expandable elastic material substantially fills said space.

5. The catheter of claim 3, wherein said outer tube and said inner tube define a space disposed between said outer tube and said inner tube, wherein said space is closed at an end of said catheter, wherein said catheter further comprises an equalization port fluidly connected to said space and a valve configured to allow pressure equalization between said space and an exterior of said catheter.

6. The catheter of claim 3, further comprising a plurality of valves each attached to an interior wall of said inner tube, wherein each of said plurality of valves is disposed in spaced relation along a length of the inner tube, wherein each valve comprises a pair of opposing flexible flaps, wherein said pair of opposing flaps of each of said plurality of valves is configured to substantially close said lumen to fluid flow through said lumen when said catheter is in said deflated configuration, and wherein said pair of opposing flaps of each of said plurality of valves is configured to separate and open said lumen to fluid flow through said lumen when said catheter is in said inflated configuration.

7. The catheter of claim 1, wherein said helical inflation tube is attached to said interior wall of said outer tube along an entire length of said helical inflation tube.

8. A catheter comprising:
a flexible outer tube;
a first helical inflation tube attached to an interior wall of said outer tube,
a second helical inflation tube attached to said interior wall of said outer tube,
wherein each helical inflation tube defines a helical fluid chamber;
an injection port,
wherein said injection port is connected to said first helical inflation tube and said second helical inflation tube at an end of said catheter,
wherein said injection port is configured to allow injection of a fluid into said first helical inflation tube and said second helical inflation tube through said injection port to transition said catheter into an inflated configuration, and wherein said injection port is further configured to allow withdrawal of said fluid from said first helical inflation tube and said second helical inflation tube through said injection port to transition said catheter into a deflated configuration,
wherein said outer tube and said helical inflation tubes define a lumen when said catheter is in said inflated configuration, and
wherein said lumen is substantially occluded when said catheter is in said deflated configuration.

9. The catheter of claim 8, wherein said first helical inflation tube and said second helical inflation tube form a double helix.

10. The catheter of claim 8, further comprising a tubular ring fluidly connected to said first helical inflation tube and said second helical inflation tube and disposed at an end of said first helical inflation tube and an end of said second helical inflation tube, wherein said injection port is connected to said tubular ring.

11. The catheter of claim 8, further comprising a flexible inner tube attached to an interior of said first helical inflation tube and said second helical inflation tube by a plurality of filaments.

12. The catheter of claim 11, wherein said outer tube and said inner tube define a space disposed between said outer tube and said inner tube, wherein said catheter further comprises an expandable elastic material disposed within said space, wherein said expandable elastic material substantially fills said space.

13. The catheter of claim 11, wherein said outer tube and said inner tube define a space disposed between said outer tube and said inner tube, wherein said space is closed at an end of said catheter, wherein said catheter further comprises an equalization port fluidly connected to said space and a valve configured to allow pressure equalization between said space and an exterior of said catheter.

14. The catheter of claim 11, further comprising a plurality of valves each attached to an interior wall of said inner tube, wherein each of said plurality of valves is disposed in spaced relation along a length of the inner tube, wherein each valve comprises a pair of opposing flexible flaps, wherein said pair of opposing flaps of each of said plurality of valves is configured to substantially close said lumen to fluid flow through said lumen when said catheter is in said deflated configuration, and wherein said pair of opposing flaps of each of said plurality of valves is configured to separate and open said lumen to fluid flow through said lumen when said catheter is in said inflated configuration.

15. The catheter of claim 8, wherein said first helical inflation tube and said second helical inflation tube are attached to said interior wall of said outer tube along an entire length of said first helical inflation tube and an entire length of said second helical inflation tube.

16. A catheter comprising:
a flexible outer tube;
a first helical inflation tube attached to an interior wall of said outer tube,
wherein said first helical inflation tube defines a first helical fluid chamber;
a second helical inflation tube attached to said interior wall of said outer tube,
wherein said second helical inflation tube defines a second helical fluid chamber;
a first injection port,
wherein said first injection port is connected to said first helical inflation tube at an end of said catheter,
wherein said first injection port is configured to allow injection of a fluid into said first helical inflation tube through said first injection port to transition said catheter into an inflated configuration, and wherein said first injection port is further configured to allow withdrawal of said fluid from said first helical inflation tube through said first injection port to transition said catheter into a deflated configuration,
a second injection port,
wherein said second injection port is connected to said second helical inflation tube at an end of said catheter,
wherein said second injection port is configured to allow injection of a fluid into said second helical inflation tube through said second injection port to transition said catheter into said inflated configuration, and wherein said second injection port is further configured to allow withdrawal of said fluid from said second helical inflation tube through said second injection port to transition said catheter into said deflated configuration,
wherein said outer tube and said helical inflation tubes define a lumen when said catheter is in said inflated configuration, and
wherein said lumen is substantially occluded when said catheter is in said deflated configuration.

17. The catheter of claim 16, further comprising a flexible inner tube attached to an interior of said first helical inflation tube and said second helical inflation tube by a plurality of filaments.

18. The catheter of claim 16, wherein said outer tube and said inner tube define a space disposed between said outer tube and said inner tube, wherein said catheter further comprises an expandable elastic material disposed within said space, wherein said expandable elastic material substantially fills said space.

19. The catheter of claim 16, wherein said outer tube and said inner tube define a space disposed between said outer tube and said inner tube, wherein said space is closed at an end of said catheter, wherein said catheter further comprises an equalization port fluidly connected to said space and a valve configured to allow pressure equalization between said space and an exterior of said catheter.

20. The catheter of claim 16, further comprising a plurality of valves each attached to an interior wall of said inner tube, wherein each of said plurality of valves is disposed in spaced relation along a length of the inner tube, wherein each valve comprises a pair of opposing flexible flaps, wherein said pair of opposing flaps of each of said plurality of valves is configured to substantially close said lumen to fluid flow through said lumen when said catheter is in said deflated configuration, and wherein said pair of opposing flaps of each of said plurality of valves is configured to separate and open said lumen to fluid flow through said lumen when said catheter is in said inflated configuration.
